(19) United States
(12) Patent Application Publication
(10) Pub. No.: US 2006/0172945 A1
(43) Pub. Date: Aug. 3, 2006

(54) Human neutrophil alpha-defensin 4 inhibition of HIV-1

(76) Inventors: Wuyuan Lu, Ellicott City, MD (US); Fiorenza Cocchi, Baltimore, MD (US); Zhibin Wu, Baltimore, MD (US)

Correspondence Address: INTELLECTUAL PROPERTY / TECHNOLOGY LAW, PO BOX 14329, RESEARCH TRIANGLE PARK, NC, 27709, US

(21) Appl. No.: 11/347,538

(22) Filed: Feb. 3, 2006

Related U.S. Application Data
(60) Provisional application No. 60/649,873, filed on Feb. 3, 2005.

(51) Int. Cl. A61K 38/16; A61K 31/7076; A61K 31/551; A61K 31/513; A61K 31/522

(52) U.S. Cl. 514/12; 514/220; 514/47; 514/263.31; 514/269

(57) ABSTRACT

A method to reduce replication of HIV-1, involving administering a therapeutically effective amount of recombinant HNP4 to a subject in need thereof to combat HIV-1 infection. The HNP4 agent may be utilized in pharmaceutical compositions including a pharmaceutically acceptable carrier and an anti-viral agent, e.g., an anti-viral agent, or combination of such agents, such as nucleoside RT inhibitors, CCR5 inhibitors/antagonists, viral entry inhibitors, and functional analogs thereof.

### CROSS-REFERENCE TO RELATED APPLICATION

The benefit of priority of U.S. Provisional Patent Application No. 60/649,873 filed Feb. 3, 2005 in the names of Wuyan Lu, et al. for “Human Neutrophil α-Defensin 4 Inhibits HIV-1 In Vitro” is hereby claimed under the provisions of 35 USC 119(e). The disclosure of said U.S. Provisional Patent Application No. 60/649,873 is hereby incorporated herein by reference, in its entirety, for all purposes.

### BACKGROUND OF THE INVENTION

1. Field of Invention

The present invention relates to α-defensins 4, including HNP1, and compositions and methods of use to inhibit replication of HIV-1.

2. Description of the Related Art

Human defensins are a family of 3-5 kDa, cationic and Cys-rich antimicrobial proteins expressed predominantly in leukocytes and epithelial cells (recent reviews: References [1-3]). They kill a broad range of microbes presumably through disruption of the negatively charged microbial membranes, playing critical roles in phagocytosis and in mucosal protection against invading pathogens.

While an important component of innate immunity, human defensins also function as effective immune modulators in adaptive immunity by chemoattracting subsets of T lymphocytes and immature dendritic cells, and, by activating receptor-mediated signaling for dendritic cell maturation.

Recently, three human neutrophil α-defensins (HNPs 1-3), differing from each other by a single amino acid residue at the N-terminus, have been implicated in suppressing HIV-1 infection in vivo [4]. However, the antiviral property of the distantly related fourth member, HNP4 [5-7], has not been described.

The dearth of prior research on HNP4 likely stems from the difficulty producing sufficient quantities of the antimicrobial protein due to the following reasons: (1) HNP4 is much less abundant in neutrophils than its three predecessors HNPs 1-3, making it difficult to purify from natural sources; (2) Recombinant HNP4 is difficult to produce due to its inherent antibiotic and membranolytic properties; (3) Chemical synthesis of HNP4, in spite of its small size (33 amino acid residues), is technically challenging due to known problems associated with defensin in vitro oxidative folding [8,9]; and (4) Reduced and unfolded HNP4 is incredibly difficult to handle chromatographically (unpublished results).

Thus, a method for generating a robust synthetic approach to, and an efficient folding protocol for, the production of human α-defensins in high purity and yield is required to generate sufficient quantities of HPN-4 to carry out comparative studies on the biological functions of this antimicrobial protein, and for production of same as a viable therapeutic agent.

### SUMMARY OF THE INVENTION

The present invention relates to HIV-1 inhibitors. In various specific aspects, the invention relates to an effective inhibitor of HIV-1 infection and use thereof to reduce replication of HIV-1.

In one aspect, the present invention relates to a method to reduce replication of HIV-1 by administering a therapeutically effective amount of recombinant HNP4. We have found that HNP4 is a significantly more effective inhibitor than HNPs 1-3 of HIV-1 infection in human peripheral mononuclear cells (PBMC) in vitro, evidencing the utility of HNP4 as a therapeutic agent for combating HIV-1 infection.

In another aspect, the present invention relates to a pharmaceutical composition comprising HNP4 and a pharmaceutically acceptable carrier.

In still another aspect, the present invention relates to a pharmaceutical composition comprising recombinant HNP4, a pharmaceutically acceptable carrier and an antiviral agent. The antiviral agent can be of any suitable type, and for example can be selected from among:

nucleoside RT inhibitors, such as Zidovudine (ZDV, AZT), Lamivudine (3TC), Stavudine (d4T), Didanosine (ddI), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);

CCR5 inhibitors/antagonists, such as SCH-C, SCH-D, PR0140, TAK 779, TAK-220, RANTES analogs, AK602, UK-427, 857, monoclonal antibodies;

viral entry inhibitors, such as Fuzeon (T-20), NB-2, NB-64, T-649, T-1249, SCH-C, SCH-D, PR0140, TAK 779, TAK-220, RANTES analogs, AK602, UK-427, 857; and functional analogs thereof.

Another aspect of the invention relates to a pharmaceutical composition comprising HNP4 and a pharmaceutically acceptable carrier, wherein the HNP4 comprises amino acid residues 64-96 of defensin 4.

Still another aspect of the present invention relates to a method of folding HPNs comprising:

generating an amino acid residue sequence of HPN lacking the pro-region; and folding such sequence in the presence of urea and N,N-dimethylformamide (DMF).

Other aspects, features and advantages of the invention will be apparent from the following detailed description, drawings and claims.

### BRIEF DESCRIPTION OF THE DRAWINGS

FIG. 1 is a graph of Percentage Inhibition of HIV-1 as a function of Defensin Concentration, μM for HIV-1 isolate IIIB (X4) in PBMC by HNPs 1-4, wherein -∘- is HNP1, -Δ- is HNP2, -X- is HNP3 and -•- is HNP4.

FIG. 2 is a graph of Percentage Inhibition of HIV-1 as a function of Defensin Concentration, μM for HIV-1 isolate BaL (R5) in PBMC by HNPs 1-4, wherein -∘- is HNP1, -Δ- is HNP2, -X- is HNP3 and -•- is HNP4.

FIG. 3 is a graph of Percentage Inhibition of HIV-1 as a function of Defensin Concentration, μM for HIV-1 isolate B703 (X4) in PBMC by HNPs 1-4, wherein -∘- is HNP1, -Δ- is HNP2, -X- is HNP3 and -•- is HNP4.

FIG. 4 is a graph of Percentage Inhibition of HIV-1 as a function of Defensin Concentration, μM for HIV-1 isolate NSI03 (R5) in PBMC by HNPs 1-4, wherein -∘- is HNP1, -Δ- is HNP2, -X- is HNP3 and -•- is HNP4.

### DETAILED DESCRIPTION OF THE INVENTION

The disclosures of the following references are hereby incorporated herein by reference:
“From Pro Defensins to Defensins: Synthesis and Characterization of Human Neutrophil Pro α-Defensins-1 and Its Mature Domain,” Z. Wu, et al., Journal of Peptide Research, Volume 62, Issue 2, Page 53-August 2003;
“Productive Folding of Human Neutrophil α-Defensins In Vitro Without the Pro-Peptide,” Z. Wu, et al., J. Am. Chem. Soc., 125 (9), 2402-2403, 2003;
“Synthesis and Characterization of Human α-Defensins 4-6,” Z. Wu, et al., The Journal of Peptide Research, Volume 64, Issue 3, Page 118-September 2004; and
U.S. Provisional Patent Application No. 60/649,873 filed Feb. 3, 2005, in the names of W. Lu, et al. for “Human Neutrophil α-Defensins 4 Inhibits HIV-1 In Vitro.”

The present invention provides a method to reduce replication of HIV-1, involving administration of a therapeutically effective amount of recombinant HNP4.

In one aspect, the invention relates to a pharmaceutical composition comprising HNP4 and a pharmaceutically acceptable carrier.

In another aspect, the present invention contemplates a method of treating a subject to reduce replication of HIV-1 in such subject, comprising administering to such subject an effective amount of HNP4, as therapeutically effective to reduce replication of HIV-1 in said subject, in relation to replication of HIV-1 in such subject in the absence of such therapeutic intervention involving the HNP4 therapeutic agent.

The subjects to be treated by such method include human subjects.

Depending on the specific level of HIV-1 infection of such subject, the subject may be administered HNP4 in the pharmaceutical composition, at any suitable therapeutically effective and safe dosage, as may readily determined within the skill of the art, and without undue experimentation.

In general, while the effective dosage of the HNP4 therapeutic agent may be widely varied in the broad practice of the invention, depending on the specific level of infection of HIV-1 in the subject, as readily determinable within the skill of the art, suitable therapeutic doses of HNP4 for achievement of therapeutic benefit may in various specific instances be in a range of 1 microgram (μg) to 100 milligrams (mg) per kilogram of the subject per day, e.g. in a range of 5 μg to 75 mg per kilogram body weight per day, or alternatively in another embodiment in a range of 10 μg to 75 mg per kilogram body weight per day, or alternatively in a further embodiment in a range of 10 μg to 50 mg per kilogram body weight per day.

Such dose may be presented as a single dose or two or more sub-doses administered at the appropriate intervals, and the sub-doses may be administered in unit dosage forms, for example, containing from 10 μg to 1,000 mg, such as from 50 μg to 500 mg, or 50 μg to 250 mg, or from 50 μg to 10 mg per unit dosage form, in various embodiments. Alternatively, if the condition of the subject so requires, the doses may be administered as an infusion.

The mode of administration and dosage forms will of course affect the therapeutic amount of the HNP4 therapeutic agent that is desirable and efficacious for the given treatment application. The administrative methods include any suitable methods such as parenteral administration, oral administration, intrathecal administration, pulmonary administration, etc., as appropriate to a specific therapeutic regimen. Specific administration modalities include: oral, rectal, topical, nasal, ophthalmic, subcutaneous, intramuscular, intravenous, transdermal, spinal, intra-articular, intra-arterial, sub-arachnoid, sub-lingual, oral mucosal, bronchial, lymphatic and intra-uterine.

Depending on the administration modality, the pharmaceutical composition may be provided in liquid solution or suspension form, or as a powered solid, for administration.

In some applications it may be advantageous to utilize the HNP4 in a “vectorized” form, such as by encapsulation in a liposome or other encapulsant medium, or by fixation of the active agent, e.g. by covalent bonding, chelation, or associative coordination, on a suitable biomolecule.

The pharmaceutical composition may therefore be formulated in any suitable manner, within the skill of the art.

In one embodiment, the invention contemplates a pharmaceutical composition comprising recombinant HNP4, a pharmaceutically acceptable carrier and an anti-viral agent. The anti-viral agent may be of any suitable type, as described more fully hereinafter.

In another composition, the invention contemplates a formulation including HNP4 and a pharmaceutically acceptable carrier, in which the HNP4 comprises amino acid residues 64-96 of defensin 4.

In the pharmaceutical compositions including HNP4, the pharmaceutically acceptable carrier may include any suitable carrier components or formulations, which are pharmaceutically acceptable in the sense of being compatible with the other ingredients of the formulations and not unduly deleterious to the subject.

In another aspect, the invention contemplates a method for folding HPNs, comprising generating an amino acid sequence of HPN lacking a pro-region, and folding such sequence in the presence of urea and N,N-dimethylformamide(DMF).

Inhibition of HIV-1 in vitro by α-defensins 1-3 was first reported by Nakashima et al, 1993 [11], which has been confirmed by several recent reports [4, 12]. To demonstrate that synthetic HNPs are active against HIV-1 infection in vitro, we tested HNPs for their inhibition of two laboratory isolates HIV_{IIIB} (X4) and HIV_{BaL} (R5) and two primary isolates B703 (X4) and NSI03 (R5) in PBMC using our standard assay protocols.

Human neutrophil α-defensins (HNPs) are small, Cys-rich, cationic antimicrobial proteins. Stored in the azurophilic granules of neutrophils, they are released during phagocytosis to kill ingested foreign microbes through disruption of their cytoplasmic membranes. HNPs are synthesized as inactive precursors in vivo and activated through proteolytic removal of their inhibitory N-terminal pro-peptide required for correct subcellular sorting and processing. Folding of HNPs in vitro without the pro-peptide has been reported to be extremely difficult which led to the hypothesis that the 45-residue anionic pro-peptide may assist proHNPs folding as an intramolecular chaperone interacting with the cationic C-terminal domain, a mechanism reminiscent of some bacterial serine proteases.

However, we have shown that HNPs without the pro-region can fold productively with yields over 80% in the presence of 2 M urea and 25% N,N-dimethylformamide (DMF). Our finding demonstrates an efficient protocol for the production of large quantities of highly pure human α-defensins and is broadly applicable in folding aggregation-prone, Cys-rich proteins of both synthetic and recombinant origin.

Human α-defensin 4, i.e., HNP4, can be synthesized as described in “Synthesis and Characterization of Human α-defensins 4-6,” Z. Wu, The Journal of Peptide Research, Volume 64, Issue 3, Page 118-September 2004, the description of which is incorporated herein in its entirety for all purposes.

A method for generating properly folded HNP4 is described in Zhibin Wu, Robert Powell, and Wuyuan Lu Am. Chem. Soc., 125 (9), 2402-2403, (2003), “Productive Folding of Human Neutrophil α-Defensins in Vitro without the Pro-peptide” the description of which hereby is incorporated by reference herein for all purposes.

Folded HNPs are known to adopt a three-stranded anti-parallel β sheet conformation constrained by three intramolecular disulfide bonds. They are highly soluble in aqueous solution and are structurally stable even in 8 M urea. In contrast, the reduced form quantitatively aggregates in the absence of high concentrations of denaturants.

Consequently, massive precipitation occurs as denaturing conditions are removed during the folding of HNPs in aqueous solution. Currently, the most effective protocol for folding HNPs without the pro-peptide utilizes the irreversible and nondiscriminating oxidant DMSO and typically results in 10% recovery. Due to the exceptionally high stability of HNPs, the prevention of aggregation of unfolded proteins, by using sufficient amounts of denaturants, suitable organic cosolvents, or both, may facilitate productive thiol-disulfide exchanges in the presence of oxidized/reduced thiol pairs, thus leading to the formation of a stable native structure. Nonetheless, we do not wish to be bound by any theory or mechanism, as regards the specific basis or reasons for such exceptionally high stability.

### EXAMPLE 1

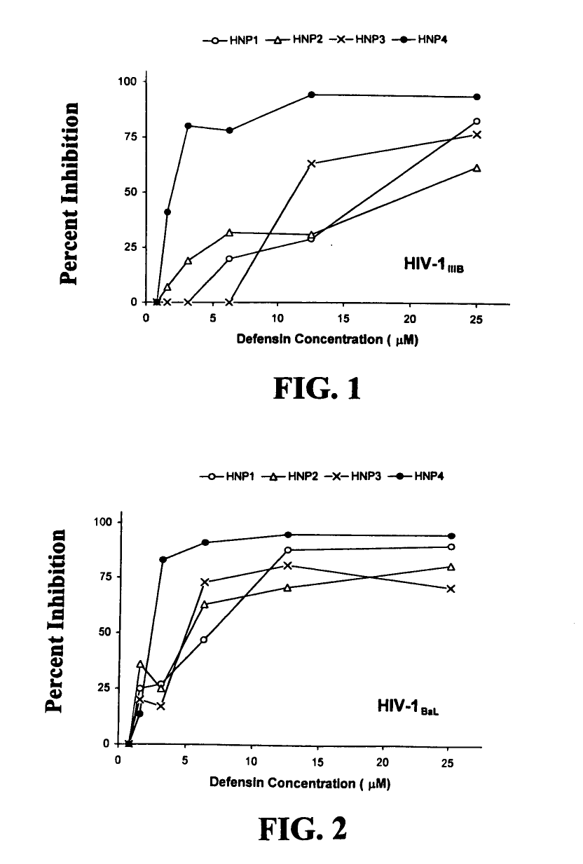

Cells infected with the HIV-1 virus were added to duplicate series of wells containing varying concentrations of test defensin in 200 ul complete culture medium, and fed after 48 hours with fresh defensin of appropriate quantities. Medium alone was used as the control. The level of HIV-1 replication was measured at day 4 after infection by HIV-1 p24 antigen-capture ELISA. Percent infection was determined as a function of HIV p24 released into the medium in sample wells versus in control wells. The data are shown in FIGS. 1-4.

FIG. 1 is a graph of Percentage Inhibition of HIV-1 as a function of Defensin Concentration, μM for HIV-1 isolate IIIB (X4) in PBMC by HNPs 1-4, wherein -∘- is HNP1, -Δ- is HNP2, -X- is HNP3 and -•- is HNP4.

FIG. 2 is a graph of Percentage Inhibition of HIV-1 as a function of Defensin Concentration, μM for HIV-1 isolate BaL (R5) in PBMC by HNPs 1-4, wherein -∘- is HNP1, -Δ- is HNP2, -X- is HNP3 and -•- is HNP4.

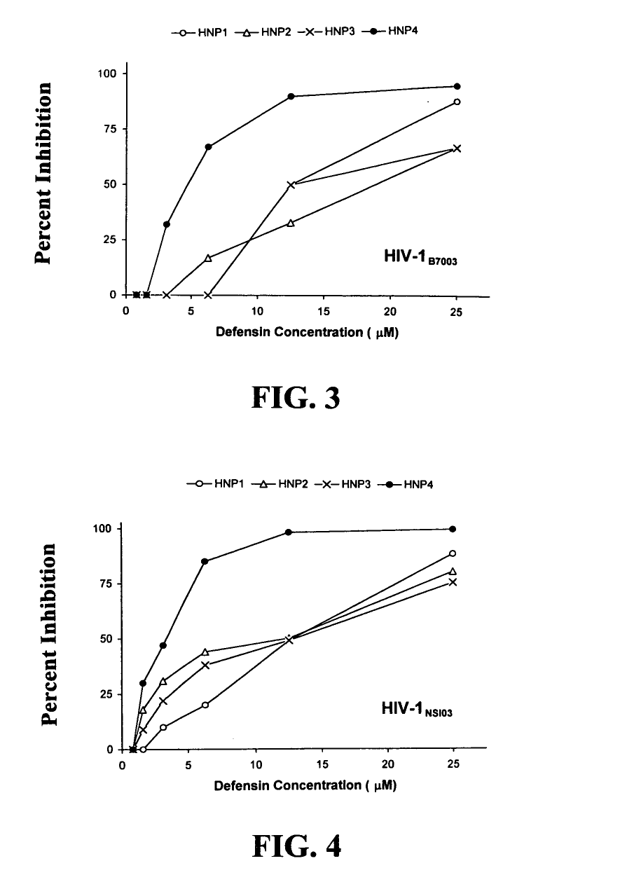

FIG. 3 is a graph of Percentage Inhibition of HIV-1 as a function of Defensin Concentration, μM for HIV-1 isolate B703 (X4) in PBMC by HNPs 1-4, wherein -∘- is HNP1, -Δ- is HNP2, -X- is HNP3 and -•- is HNP4.

FIG. 4 is a graph of Percentage Inhibition of HIV-1 as a function of Defensin Concentration, μM for HIV-1 isolate NSI03 (R5) in PBMC by HNPs 1-4, wherein -∘- is HNP1, -Δ- is HNP2, -X- is HNP3 and -•- is HNP4.

All four α-defensins inhibit their susceptible HIV-1 strains in a dose-dependent manner, with similar IC₅₀ values for HNPs 1-3 in the range of 5-20 uM. However, inhibition of both strains of HIV-1 by HNP4 is significantly and unexpectedly stronger as compared with that achieved by HNPs 1-3.

### REFERENCES

1. Lehrer, R. I., and Ganz, T. (2002) Defensins of vertebrate animals. Curr Opin Immunol 14, 96-102.
2. Zasloff, M. (2002) Antimicrobial peptides of multicellular organisms. Nature 415, 389-395.
3. Ganz, T. (2003) Defensins: antimicrobial peptides of innate immunity. Nat Immunol 3, 710-720.
4. Zhang, L., Yu, W., He, T., Yu, J., Caffrey, R. E., Dalmasso, E. A., Fu, S., Pham, T., Mei, J., Ho, J. J., Zhang, W., Lopez, P., and Ho, D. D. (2002) Contribution of human alpha-defensin 1, 2, and 3 to the anti-HIV-1 activity of CD8 antiviral factor. Science 298, 995-1000.
5. Wilde, C. G., Griffith, J. E., Marra, M. N., Snable, J. L., and Scott, R. W. (1989) Purification and characterization of human neutrophil peptide 4, a novel member of the defensin family. J Biol Chem 264, 11200-11203.
6. Gabay, J. E., Scott, R. W., Campanelli, D., Griffith, J., Wilde, C., Marra, M. N., Seeger, M., and Nathan, C. F. (1989) Antibiotic proteins of human polymorphonuclear leukocytes. Proc Natl Acad Sci USA 86, 5610-5614.
7. Singh, A., Bateman, A., Zhu, Q. Z., Shimasaki, S., Esch, F., and Solomon, S. (1988) Structure of a novel human granulocyte peptide with anti-ACTH activity. Biochem Biophys Res Commun 155, 524-529.
8. Raj, P. A., Antonyraj, K. J., and Karunakaran, T. (2000) Large-scale synthesis and functional elements for the antimicrobial activity of defensins. Biochem J 347 Pt 3, 633-641.
9. Tam, J. P., Wu, C. R., Liu, W., and Zhang, J. W. (1991) Disulfide bond formation in peptides by dimethyl-sulfoxide—scope and applications. J Am Chem Soc 113, 6657-6662.
10. Wu, Z., Powell, R., and Lu, W. (2003) Productive folding of human neutrophil α-defensins in vitro without the pro-peptide. J Am Chem Soc 125, 2402-2403.
11. Nakashima, H., Yamamoto, N., Masuda, M., and Fujii, N. (1993) Defensins inhibit HIV replication in vitro. AIDS 7, 1129.
12. Chang, T. L., Francois, F., Mosoian, A., and Klotman, M. E. (2003) CAF-mediated human immunodeficiency virus (HIV) type 1 transcriptional inhibition is distinct from alpha-defensin-1 HIV inhibition. J Virol 77, 6777-6784.

What is claimed is:

1. A method to reduce replication of HIV-1, the method comprising:
administering a therapeutically effective amount of recombinant HNP4.

2. A pharmaceutical composition comprising HNP4 and a pharmaceutically acceptable carrier.

3. A pharmaceutical composition comprising recombinant HNP4, a pharmaceutically acceptable carrier and an antiviral agent.

4. A pharmaceutical composition comprising HNP4 and a pharmaceutically acceptable carrier, wherein the HNP4 comprises amino acid residues 64-96 of defensin 4.

5. The pharmaceutical composition according to claim 3, wherein the antiviral agent is selected from the group consisting of nucleoside RT inhibitors, CCR5 inhibitors/antagonists, viral entry inhibitors and functional analogs thereof.

6. The pharmaceutical composition according to claim 3, wherein the antiviral agent comprises a nucleoside RT inhibitor selected from the group consisting of Zidovudine (ZDV, AZT), Lamivudine (3TC), Stavudine (d4T), Didanosine (ddI), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), and Trizivir (ZDV/3TC/ABC), and functional analogs of the foregoing.

7. The pharmaceutical composition according to claim 3, wherein the antiviral agent comprises a CCR5 inhibitor/antagonists selected from the group consisting of SCH-C, SCH-D, PR0140, TAK 779, TAK-220, RANTES analogs, AK602, UK-427, 857, monoclonal antibodies, and functional analogs of the foregoing.

8. The pharmaceutical composition according to claim 3, wherein the antiviral agent comprises a viral entry inhibitor selected from the group consisting of Fuzeon (T-20), NB-2, NB-64, T-649, T-1249, SCH-C, SCH-D, PRO 140, TAK 779, TAK-220, RANTES analogs, AK602, UK-427, 857, and functional analogs thereof.

9. A method for folding HPNs, comprising:
generating an amino acid residue sequence of HPN lacking a pro-region; and
folding such sequence in the presence of urea and NN-dimethylformamide (DMF).
